(19) United States
(12) Patent Application Publication
(10) Pub. No.: US 2023/0072854 A1
(43) Pub. Date: Mar. 9, 2023

(54) PARAXANTHINE-BASED BIOACTIVE COMPOSITION AND METHOD OF USE THEREOF

(71) Applicant: Ingenious Ingredients, LP, Lewisville, TX (US)

(72) Inventors: Martin Purpura, Spring, TX (US); Shawn D. Wells, Frisco, TX (US); Ralf Jäger, Whitefish Bay, WI (US); Kylin Liao, Plano, TX (US)

(21) Appl. No.: 17/794,729

(22) PCT Filed: Jan. 25, 2021
(86) PCT No.: PCT/US21/14958

§ 371 Date: Jul. 22, 2022

Related U.S. Application Data
(60) Provisional application No. 62/964,976, filed on Jan. 23, 2020.

(51) Int. Cl. A61K 31/522; A61K 45/06; A61P 25/00

(52) U.S. Cl. CPC A61K 31/522; A61K 45/06; A61P 25/00

(57) ABSTRACT

The disclosed compositions, systems and methods relate to a dietary supplement for human consumption and comprises paraxanthine and optionally other compounds that modulate the effects of paraxanthine. Uses for the paraxanthine-containing supplements contain improvement of at least one of endurance performance, mood, vigor, lipolysis, energy expenditure, exercise performance, and/or decreased appetite.

### CROSS-REFERENCE TO RELATED APPLICATION(S)

This application claims priority to U.S. Patent Appln. No. 62/964,976, filed Jan. 23, 2020 and entitled “PARAXANTHINE-BASED BIOACTIVE COMPOSITION AND METHOD OF USE THEREOF,” which is hereby incorporated by reference in its entirety.

### TECHNICAL FIELD

The disclosed technology relates generally to compositions, methods, and system for utilizing paraxanthine alone and in combination for use in providing physiological benefits. More particularly, the disclosure relates to paraxanthine and other compounds, whether produced synthetically or derived from natural sources, and use of these chemical compounds to provide physiological benefits, which may vary according to paraxanthine concentration and the presence of synergists and antagonists.

### BACKGROUND

Paraxanthine, also known as 1,7-dimethylxanthine or 1,7-Dimethyl-3H-purine-2,6-dione, is a dimethyl derivative of xanthine, structurally related to caffeine as well as a metabolite of caffeine, accounting for 80% of the total caffeine excretion in humans. In humans and other animals caffeine is first degraded to either paraxanthine (1,7-dimethylxanthine), theobromine or theophylline. Paraxanthine is observed in nature as a metabolite of caffeine in animals and humans. Paraxanthine is also found naturally occurring in various plant species, such as Citrus paradisi (grapefruit), Theobroma cacao (cocoa) and Camilla sinensis (tea).

Caffeine is a bitter, white crystalline purine, a methylxanthine alkaloid, and is chemically related to the adenine and guanine bases of deoxyribonucleic acid (DNA) and ribonucleic acid (RNA). It is found in the seeds, nuts, or leaves of a number of plants native to Africa, East Asia and South America, and helps to protect them against predator insects and to prevent germination of nearby seeds. The most well-known source of caffeine is the coffee bean, a misnomer for the seed of Coffea plants.

Caffeine is by far the most studied, and the most commonly used stimulant found in coffee and tea products. Paraxanthine appears to have an improved effect over caffeine, despite being very similar in chemical structure. Recent experiments have shown that paraxanthine exhibits a variety of activities, some of which seem inconsistent.

Concerns about potential health risks of coffee and caffeine consumption raised by epidemiological research in the past were likely exacerbated by associations between high intakes of coffee and unhealthy behaviors, such as cigarette smoking and physical inactivity. More recently, coffee consumption has been associated with reductions in the risk of several chronic diseases.

Caffeine concentrations in coffee beverages can be quite variable. A standard cup of coffee is often assumed to provide 100 mg of caffeine, but a recent analysis of 14 different specialty coffees purchased at coffee shops in the US found that the amount of caffeine in 8 oz (˜240 ml) of brewed coffee ranged from 72-130 mg (McCusker, R. R., Goldberger, B. A. and Cone, E. J. 2003. Caffeine content of specialty coffees. J. Anal. Toxicol., 27: 520-522.). Caffeine in espresso coffees ranged from 58-76 mg in a single shot. Interestingly, the caffeine content of the same type of coffee purchased from the same store on six separate days varied from 130 to 282 mg per 8-oz serving.

Thus, there is a need in the art to identify alternative chemical compounds and mixtures thereof that may provide benefits. It is also desirable to provide chemical compounds and mixtures thereof that may be used to provide a variety of benefits, varying by concentration, thus requiring production of fewer materials.

### BRIEF SUMMARY

This disclosure relates to the use of a chemical composition comprising paraxanthine, either naturally or synthetically produced, and optionally other chemicals, including paraxanthine congeners or analogs, to provide a plurality of desirable effects. Paraxanthine analogs may include, but are not limited to, caffeine, methyl caffeine, theobromine, theophylline, liberine and methylliberine, and their variants. Other suitable actives may include one or more ergogenic or nootropic compounds such, St John's Wort, sulbutiamine, and the like.

Paraxanthine exhibits a wide variety of effects depending on dosage. The presence of other ingredients may also modulate its effects. It may be used to improve endurance performance, mood, promote calm and focus, vigor, lipolysis, energy expenditure, exercise performance, and/or decreased appetite. It may also serve as an antioxidant and an anti-inflammatory.

In one embodiment, paraxanthine may be used to modulate stimulants, to provide heightened energy without heightened anxiety or nervousness. There may be variability among individuals, as described herein.

In another embodiment paraxanthine may be used as a mild mood enhancer or relaxant.

In a further embodiment, paraxanthine may be used to promote weight loss by reducing appetite, act as an antioxidant and as an anti-inflammatory. Paraxanthine may be used transdermally to enhance one or more of these effects.

In one embodiment, a dietary supplement comprising about 2 mg to about 800 mg paraxanthine, either natural through fermentation or synthetic, is provided.

In another embodiment, a method of treatment for improving physical performance or energy in an individual is provided. This method involves providing the individual with a composition comprising about 2 mg to about 800 mg of paraxanthine, either natural or synthetic, wherein upon administration of the composition the individual experiences improvement of at least one of endurance performance, mood, promote calm and focus, vigor, lipolysis, energy expenditure, exercise performance, and/or decreased appetite. In another embodiment, a second compound such as caffeine may also be administered in the composition.

It is therefore an object of the present disclosure to provide compositions including paraxanthine capable of imparting a plurality of positive effects.

It is another object of the present disclosure to provide congeners, derivatives and iterations of paraxanthine and synthetic chemical equivalents of paraxanthine.

It is another object of the present disclosure to provide agglomerated paraxanthine, paraxanthine salts, microencapsulated, liposomal or esterified paraxanthine.

It is another object of the present disclosure to provide paraxanthine combined with glycerides, propylene glycol, polyethylene glycol (PEG), lauroyl macrogol, lauroyl macrogol derivatives and co-crystallization products of paraxanthine.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE FIGURES

FIG. 1 shows correct responses to BCST test.

FIG. 2 shows errors on BSCT test.

FIG. 3 shows pervasive errors (PEBL) on BSCT test.

FIG. 4 shows pervasive errors (PAR) on BSCT test.

FIG. 5 shows correct response mean accuracy on Go/No-Go test.

FIG. 6 shows Round 1 Condition P mean response time on Go/No-Go test.

FIG. 7 shows Round 1 Condition R mean response time on Go/No-Go test.

FIG. 8 shows Round 2 Condition P mean response time on Go/No-Go test.

FIG. 9 shows Round 2 Condition R means response time on Go/No-Go test.

FIG. 10 shows average response time for Go/No-Go test.

FIG. 11 shows Trial #2 reaction time for vigilance test.

FIG. 12 shows Trial #10 reaction time for vigilance test.

FIG. 13 shows Trial #20 reaction time for vigilance test.

FIG. 14 shows average reaction time for vigilance test.

FIG. 15 shows letter length 2 absent reaction time on Sternberg test.

FIG. 16 shows letter length 4 absent reaction time on Sternberg test.

FIG. 17 shows letter length 6 absent reaction time on Sternberg test.

FIG. 18 shows letter length 2 present reaction time on Sternberg test.

FIG. 19 shows letter length 4 present reaction time on Sternberg test.

FIG. 20 shows letter length 6 present reaction time on Sternberg test.

FIG. 21 shows a bitterness scale.

FIG. 22 shows solutions, abbreviations, and concentrations.

FIG. 23 shows results of bitterness taste test.

FIG. 24 shows results of bitterness taste test on bitterness scale.

FIG. 25 shows comments from tasters from bitterness test.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause unacceptable adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Disclosed are compositions comprising paraxanthine and the related uses thereof. Paraxanthine is also known as 1,7-dimethylxanthine or 1,7-Dimethyl-3H-purine-2,6-dione. Paraxanthine may be produced synthetically or may be isolated from a natural source or through fermentation. Paraxanthine isolated from such sources may be purified to 95% or greater purity. Optionally, less purification may be used such that paraxanthine accounts for 50%, or even less, of the material. In some embodiments, it may be preferable to utilize paraxanthine isolated from a natural source which may include other congeners of paraxanthine typically found in paraxanthine sources.

In certain embodiments, paraxanthine may be combined with one or more other chemical compounds (e.g. other active ingredients), to provide a plurality of positive effects in a subject. By altering the dosage of paraxanthine and/or chemical compounds it is combined with, various physiological effects may be selected for. The compositions may provide primarily a single benefit, or may provide multiple benefits simultaneously.

In certain embodiments, paraxanthine is combined with one or more additional active ingredients selected from: a group consisting of: gallic acid, (+)-catechin (C), (−)-epicatechin (EC), (+)-gallocatechin (GC), (−)-epigallocatechin (EGC), (−)-catechin gallate (CG), (−)-gallocatechin gallate (GCG), (−)-epicatechin gallate (ECG) and (−)-epigallocatechin gallate (EGCG), glycerides, propylene glycol, lauroyl macrogol, lauroyl macrogol derivatives, co-crystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, krill oil, astaxanthin, proteolytic enzymes, glucosamine sulfate, chondroitin sulfate, MSM (methylsulfonylmethane), SAMe (S-adenosylmethionine), ASU (avocado-soybean unsapponifiable fraction), cetyl myristoleate, Dolichos falcate, triterpenoids, acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, flavenoids, tocopherols, theophylline, alpha-yohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, ginkgo Biloba, siberian ginseng, astragalus, licorice, green tea, reishi, dehydroepiandrosterone (DHEA), pregnenolone, tyrosine, N-acetyl-tyrosine, glucuronolactone, taurine, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, and forskolin (Coleus forskohlli), 2-(dimethylamino)ethanol (DMAE), DMAE bitartrate, and combinations thereof.

In another embodiment, paraxanthine may be used at lower dosage levels and/or in conjunction with compounds that modulate or antagonize its activity. Such compositions may induce an improved endurance performance, mood, vigor, lipolysis, energy expenditure, exercise performance, and/or decreased appetite.

An advantage of using the invention may be the reduced likelihood that a person develops a tolerance to chemical compositions in accordance with the principles of the invention. That is, a person may not become desensitized to the effects induced.

According to certain aspects, the disclosed paraxanthine containing compositions has at least the following distinct advantages over the administration of compositions containing comparable doses of caffeine. Paraxanthine has substantially lower toxicity. Paraxanthine has greater stability (e.g. does not lose potency over time to the same extent as caffeine). Paraxanthine containing compositions are more potent wake-promoting agent (in certain embodiments, via adenosine receptor antagonism). Further, Paraxanthine containing compositions enhance striatal dopaminergic tone. Still further, paraxanthine does not produce sleep rebound. Further, paraxanthine does not produce withdrawal effects upon cessation of use, as frequently occurs with caffeine. Yet further, paraxanthine does not enhance anxiety. Still further, paraxanthine is less bitter than caffeine. Even further, paraxanthine is effective for a larger portion of the population than caffeine.

In another embodiment, paraxanthine may be used at higher dosage levels and/or with synergistic compounds. These compositions may increase a person's basal/resting metabolic rate, increase thermogenesis, decrease appetite, enhance cognitive performance, increase Alpha wave brain activity, and/or induce euphoria. Without being bound by theory, the inventors believe that at higher dosage levels, paraxanthine may be noradrenergic and dopaminergic, and may exhibit increased adenosine receptor inhibition.

In another embodiment, paraxanthine is combined with ephedrine, caffeine, salicylic acid or the like. The foregoing combinations may produce a synergistic effect with the stimulating effects of paraxanthine. For example, in certain embodiments, paraxanthine is be combined with much lesser amounts of caffeine in order to modulate the excessive stimulatory effects of caffeine, thereby stabilizing heart rate and other metabolic activity. That is, a combination of paraxanthine and caffeine may result in a composition that imparts the increased focus and energy induced by caffeine, but without the higher heart rate and blood pressure due to modulation of caffeine's effects by paraxanthine. Thus the combination may result in heightened awareness and calmness without the jitters caffeine may cause.

According to further embodiments, dietary supplements comprising paraxanthine are used to enhance athletic performance. According to exemplary aspects of these embodiments, that may reduce fatigue, improve energy, increase mobility, and improve alertness. In further embodiments, administration of the disclose compositions is cardio protective. In further embodiments, administration of the disclose compositions improves muscle contractions and muscle performance. In exemplary aspects, of these embodiments, muscle performance is enhanced through increasing potassium (K+) transport into skeletal muscle. In further aspects, muscle performance is enhanced through increasing intracellular calcium (e.g., via ryanodine receptor (RyR) activation).

In another embodiment, paraxanthine may be used as a topical agent for incorporation into body creams or lotions to produce a cream or lotion for lightening skin, firming skin, and/or improving skin elasticity. A paraxanthine topical agent may also be used to promote localized transdermal fat loss. Paraxanthine may also be used in a cream or lotion to promote localized enhanced metabolism and/or enhanced thermogenesis.

According to further embodiments, paraxanthine is be combined with one or more of analgesics and/or anti-inflammatory agents. In exemplary implementations, paraxanthine is combined with ibuprofen, salicylic acid, anti-inflammatory agents, salicin, fish oil (omega-3 fatty acids and specialized, small lipid pro-resolving derivatives), tart cherry, krill oil, astaxanthin, proteolytic enzymes, glucosamine sulfate, chondroitin sulfate, MSM (methylsulfonylmethane), SAMe (S-adenosylmethionine), ASU (avocado-soybean unsapponifiable fraction), cetyl myristoleate, Dolichos falcate and/or triterpenoids.

The dosage of paraxanthine may range from about 2 mg to about 800 mg. In another embodiment, the range may be from about 50 mg to about 400 mg.

In another embodiment, paraxanthine is combined with one or more bioavailability enhancers. In exemplary embodiments, bioavailability enhancers include, but are not limited to: bioperine, piperine, black pepper, bergamottin, dihydroxybergamottin (CYP3A4 inhibitors), flavonoids (including hesperidin, naringin, tangeritin, quercetin and nobiletin both in isolation and in combination), pterostilbenes, fisetin, nanoencapsulation, microencapsulation, liposomes and/or phytosomes. Which enhancers are combined with paraxanthine may depend on which qualities of paraxanthine are desired for a particular use.

In another embodiment, paraxanthine may be administered using one or more delivery methods, including, for example transdermal patches and/or creams, ready to mix powders, intravenous methods, capsules, tablets, liquid (including liquids for mixing with other beverages), softgels, shot format, and/or cosmetic applications including soaps, lotions and shampoos. Paraxanthine's anti-inflammatory qualities may be desired for a variety of topical applications.

Methods of Treatment

According to certain embodiments, the composition disclosed herein are used in the treatment of one or more medical conditions in a subject in need thereof. In certain implementations, the disclosed composition is administered to a subject suffering from narcolepsy, sleep apnea, and shift work sleep disorder, insomnia epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), Parkinson's disease, Alzheimer's, and/or dementia.

In certain aspects, the disclosed compositions are a neuroprotective agent. In certain embodiments, administration of the disclosed compositions to a subject in need thereof is neuroprotective. In exemplary aspects of these embodiments, this neuroprotection is in the form of protecting against dopaminergic cell death.

According to further embodiments, disclosed compositions are useful for the treatment of geriatric depression. In exemplary embodiments, the compositions are effective in treating subjects suffering from geriatric depression an essential, vascular or traumatic origin. and of the mental decay in the elderly.

The administration of the disclosed compositions to a subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

One general aspect includes a dietary supplement may include a first active ingredient may include about 2 mg to about 800 mg paraxanthine Other embodiments of this aspect include corresponding computer systems, apparatus, and computer programs recorded on one or more computer storage devices, each configured to perform the actions of the methods.

Implementations may include one or more of the following features. The dietary supplement may include a second active ingredient, selected from a group may include of: gallic acid, (+)-catechin (c), (−)-epicatechin (ec), (+)-gallocatechin (gc), (−)-epigallocatechin (egc), (−)-catechin gallate (cg), (−)-gallocatechin gallate (gcg), (−)-epicatechin gallate (ecg) and (−)-epigallocatechin gallate (egcg), glycerides, propylene glycol, lauroyl macrogol, lauroyl macrogol derivatives, co-crystallization products of bioperine, piperine, black pepper, bergamottin, dihydroxybergamottin (cyp3a4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, hill oil, astaxanthin, proteolytic enzymes, glucosamine sulfate, chondroitin sulfate, msm (methylsulfonylmethane), same (s-adenosylmethionine), asu (avocado-soybean unsapponifiable fraction), cetyl myristoleate,dolichosfalcate, triterpenoids,acacia catechu, andrographis paniculata, scutalleria baicalensis, agmatine sulfate, stinging nettle, sea buckthorn, curcumin,cissus quadrilangularis, boswellia serrata, wasabia japonica (wasabi extract for tea tree oil), emu oil,arnica, mangifera indical. (anacardiaceae),lagenaria breviflora, zingiber Officinale(ginger & gingerols/shogaols),hoodia gordonii, caffeine, yohimbine, methylsynephrine, synephrine, theobromine, flavenoids, tocopherols, theophylline, alpha-yohimbine, conjugated linoleic acid (cla), octopamine, evodiamine, passion flower, red pepper, cayenne, raspberry ketone, guggul, green tea, guarana, kola nut, beta-phenethylamines,acacia rigidula, forskolin (coleus forskohlli), theophylline, synephrine, yohimbine,rhodiola, ashwagandha,ginseng, ginkgo Biloba, siberianginseng, astragalus, licorice, green tea, reishi, dehydroepiandrosterone (DHEA), pregnenolone, tyrosine, n-acetyl-tyrosine, glucuronolactone, taurine, acetyl-1-carnitine, 5-hydroxytryptophan, tryptophan, phenethylamines,sceletium tortuosum (and mesembrine alkaloids),dendrobiumsp.,acacia rigidula, pqq (pyroloquinoline quinone), ubiquinone(01), nicotinamide riboside, picamilon, huperzine a (chinese clubmoss orhuperzia serrata, 1-dopa,mucuna pruriens, and forskolin (coleus forskohlli), 2-(dimethylamino)ethanol (DMAE), dmae bitartrate, and combinations thereof. The dietary supplement may include a pharmaceutically acceptable carrier. The supplement is in a solid oral dosage form. The supplement is in a topical form for administration. The dietary supplement may include a paraxanthine congener or paraxanthine analog. Said paraxanthine congener or analog is selected from the group may include of caffeine, methyl caffeine, theobromine, theophylline, liberine, methylliberine, and combinations thereof. The paraxanthine congener or analog is caffeine. The effective dose of caffeine is lower than the effective dose of caffeine in a composition without paraxanthine.

One general aspect includes a method for improving physical performance or energy in subject. The method also includes providing the subject with a composition may include about 2 mg to about 800 mg of paraxanthine, either natural or synthetic.

Implementations may include one or more of the following features. The method where upon administration of the composition, the subject experiences improvement of at least one of mood, energy, focus, concentration or sexual desire or a reduction of at least one of anxiety, fatigue, perception of effort or perception of pain. Upon continued administration to the subject, the composition does not create dependence in the subject and/or withdrawal effect in the subject when continued use is ceased. The amount of paraxanthine provided is about 50 mg to about 400 mg. The subject experiences a decrease in fatigue of at least about 6 percent. The subject experiences an increase in energy of at least about 8 percent. The composition further may include at least one ingredient selected from the group may include of caffeine, theobromine, naringin, hesperidin, 2-(dimethylamino)ethanol (DMAE), dmae bitartrate, and huperzine a.

One general aspect includes a method of treating a condition in a subject in need thereof. Implementations may include one or more of the following features. The method where the condition is selected from narcolepsy, epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), Parkinson's disease, Alzheimer's, and dementia. The paraxanthine is present from about 2 mg to about 800 mg. The composition is administered in a therapeutically effective amount. The composition is administered in a prophylactically effective amount. The composition may include paraxanthine at an amount from about 2 mg to about 800 mg. The composition may include paraxanthine at an amount from about 50 mg to about 400 mg.

One general aspect includes a method of enhancing attention in a subject in need thereof may include administering a composition to the subject may include paraxanthine.

One general aspect includes a method of improving working memory in a subject in need thereof comprising administering a paraxanthine containing composition to the subject.

One general aspect includes a method of improving cognitive performance in a subject may include administering a composition to the subject comprising paraxanthine.

One general aspect includes a method of aiding weight loss and/or fat loss in a subject may by administering a composition to the subject comprising paraxanthine.

Implementations may include one or more of the following features. The method where the weight loss results from increased metabolism in the subject. The weight loss results from decreased caloric intake in the subject.

One general aspect includes a caffeine substitute composition for use in a dietary supplements may include paraxanthine. Implementations may include one or more of the following features. The composition where the composition does not increase anxiety when administered to a subject relative to a comparable dose of caffeine. The composition does not create dependence in a subject upon repeated administrations and does not create withdrawal effects in the subject upon cessation of use. The composition where the composition is less bitter than a comparable dose of caffeine. The composition where the composition is less toxic than a comparable dose of caffeine.

### Examples

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

In a double blind, randomized, crossover, and repeated measures manner, 13 volunteers (10 male, 3 female, average age 24±5 years, average body weight 72.91±19.30 kg, average height 169.96±11.84 cm, average BMI 24.80±3.71 kg/m²) participated in this study. Participants limited ingestion of caffeine and other stimulants to normal use for 48-hours, fasted for 8-hours and reported to the lab and completed 4 different cognitive function tests to assess short-term attention and inhibitory control (Go-No Go Test), sustained attention (Vigilance Task Test), working memory (Sternberg Task Test) and cognitive flexibility (Berg-Washington Card Sorting Test). Once completed, the participants ingested either a placebo or 200 mg of paraxanthine (WGI, TX, USA) with 8 ounces of water. Participants repeated cognitive function tests 1, 2, 3, 4, 5, and 6 hours after ingestion of the supplement. After one-week participants repeated the experiment while consuming the other treatment.

Data were analyzed by a General Linear Model multivariate and univariate analyses with repeated measures using body weight as a covariate. Data are expressed as means±standard deviations for the placebo (PLA) and Paraxanthine (PrX). Greenhouse-Geisser univariate p-levels are listed for time (T), weight×time (W×T) and group×time (G×T) interactions effects. Pairwise comparison is indicated by the following superscripts: †=p<0.5 difference from baseline value; *=0.5<p<0.10 difference from baseline; a=p<0.5 difference between groups; b=0.5<p<0.10 difference between groups. Eta2 vaules of 0.01-0.5 are considered small, 0.6-0.13 medium, and >0.14 are considered large.

### Berg-Washington Card Sorting Task Test

Berg-Washington Card Sorting Task Test is a test of basic cognitive flexibility or set-shifting between old/new rule changes. The test involves reasoning, learning, executive control and attention shifting. It is particularly sensitive to the inability to shift set. Less errors means subjects were able to both recognize and ‘shift’ to a new rule with higher ability. The test involves reasoning, learning, executive control and attention shifting.

Overall, paraxanthine group had 5.3% more correct responses (placebo 102.5 vs. paraxanthine 97.3) and 23.7% less errors (placebo 23.6 vs. paraxanthine 18.0). Adjusted for differences in baseline values, paraxanthine administration resulted in 150% more correct responses (placebo 1.0 vs. paraxanthine 2.5) and 600% less errors (placebo 0.3 vs. paraxanthine −1.5). Looking at the individual results at the different timepoints (0-6 hours), paraxanthine administration resulted in improvements even after 6 hours (placebo+1.9 errors, −1.5 correct answers vs. paraxanthine −0.6 errors, +1.9 correct answers), indicating long lasting benefits of paraxanthine.

Paraxanthine significantly increased the number of correct answers, while reducing the number of errors in the Berg-Washington Card Sorting Task Test (BCST). Paraxanthine increased cognitive flexibility or set-shifting between old/new rule changes.

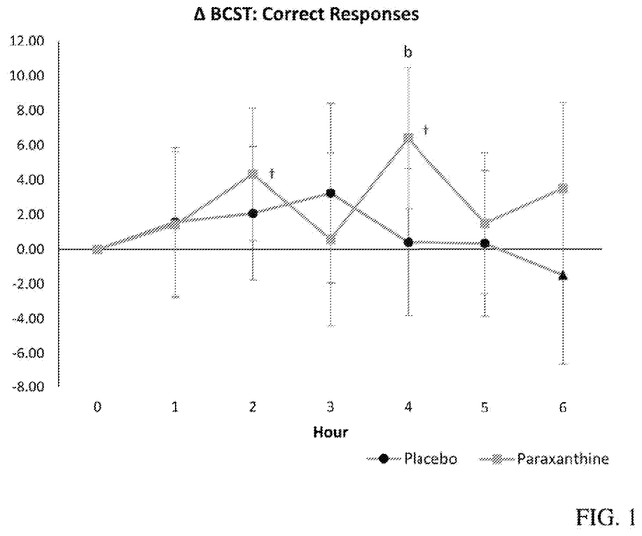

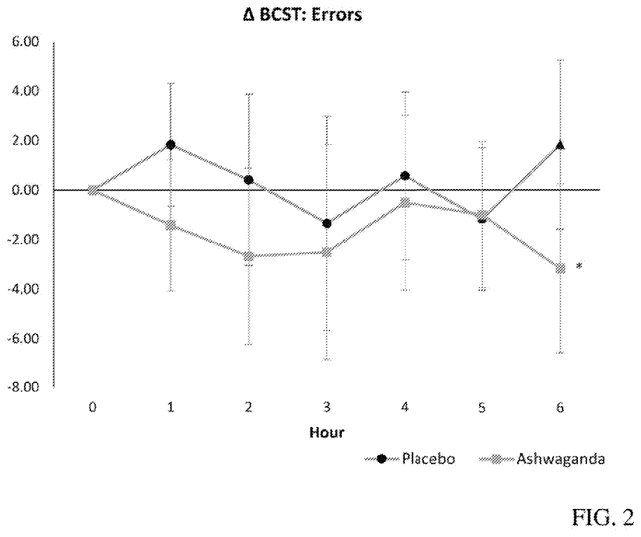

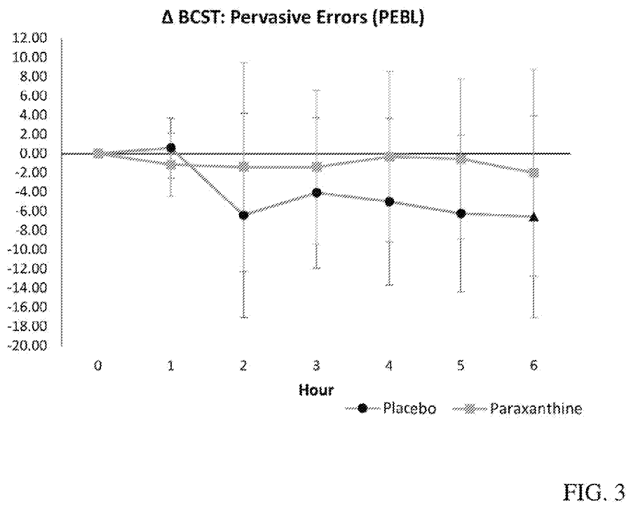

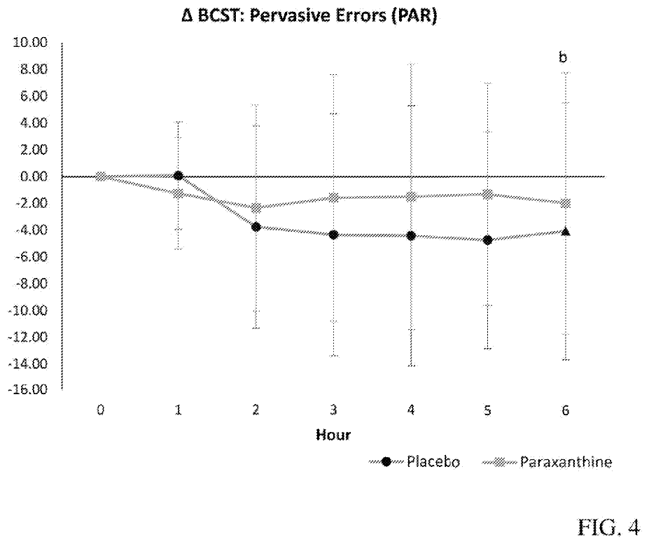

FIGS. 1-4 show various graphs of the BCST test results: FIG. 1 shows paraxanthine significantly (p<0.5) increased the number of correct responses at hour 2 and 4 over baseline, and showed a trend (p<0.1) between groups at hour 4; FIG. 2 shows paraxanthine showed a trend (p<0.1) towards less error compared to baseline at hour 6; FIG. 3 shows no significant time or between treatment effects although paraxanthine appeared to maintain PEBL to a greater degree; and FIG. 4 shows paraxanthine showed a trend (p<0.1) towards less errors between groups at hour 6.

TABLE 4
Results of the BCST Test
BCST

| | | Hour | | | | | | | | | |
|---|---|---|---|---|---|---|---|---|---|---|---|
| Variable | Group | 0 | 1 | 2 | | 3 | 4 | | 5 | 6 | |
| Correct Response | PLA | 96.3 ± 19.2 | 97.9 ± 16.0 | 98.4 ± | 15.7 | 99.6 ± 12.5 | 96.8 ± | 15.7 | 96.7 ± 17.0 | 94.8 ± | 22.3 |
| | PrX | 100.0 ± 6.6 | 101.4 ± 3.6 | 104.3 ± | 4.2 ^{†} | 100.6 ± 5.8 | 106.4 ± | 2.0 ^{†} | 101.5 ± 4.0 | 103.5 ± | 6.1 |
| | Time | 98.2 ± 14.2 | 99.7 ± 11.5 | 101.4 ± | 11.7 ^{†} | 100.1 ± 9.5 | 101.6 ± | 12.0 ^{b†} | 99.1 ± 12.3 | 99.2 ± | 16.6 |
| Errors | PLA | 23.3 ± 21.2 | 25.2 ± 18.1 | 23.8 ± | 17.3 | 22.0 ± 14.1 | 23.9 ± | 17.9 | 22.2 ± 19.3 | 25.2 ± | 24.3 |
| | PrX | 19.5 ± 6.6 | 18.1 ± 4.3 | 16.8 ± | 2.9 | 17.0 ± 3.9 | 19.0 ± | 2.4 | 18.5 ± 4.2 | 16.3 ± | 3.1 * |
| | Time | 21.4 ± 15.5 | 21.6 ± 13.4 | 20.3 ± | 12.6 | 19.5 ± 10.4 | 21.5 ± | 12.7 | 20.3 ± 13.8 | 20.8 ± | 17.6 |
| Perseverative Errors | PLA | 18.3 ± 20.9 | 18.9 ± 15.6 | 11.9 ± | 4.9 | 14.3 ± 3.7 | 13.3 ± | 3.7 | 12.1 ± 3.5 | 11.8 ± | 4.7 |
| (PEBL) | PrX | 13.7 ± 5.0 | 12.5 ± 3.0 | 12.3 ± | 2.9 | 12.3 ± 3.2 | 13.3 ± | 2.2 | 13.1 ± 3.6 | 117 ± | 2.6 |
| | Time | 16.0 ± 15.0 | 15.7 ± 11.5 | 12.1 ± | 3.9 | 13.3 ± 3.5 | 13.3 ± | 3.0 | 12.6 ± 3.5 | 11.7 ± | 3.7 |
| Perseverative Errors | PLA | 14.0 ± 23.1 | 14.1 ± 14.4 | 10.3 ± | 6.4 | 9.7 ± 3.1 | 9.6 ± | 2.5 | 9.3 ± 5.0 | 9.9 ± | 2.8 |
| (PAR rules) | PrX | 10.1 ± 4.6 | 8.8 ± 2.1 | 7.8 ± | 2.5 | 8.5 ± 2.9 | 8.6 ± | 2.4 | 8.8 ± 3.6 | 8.1 ± | 1.5 |
| | Time | 12.0 ± 16.4 | 11.5 ± 10.4 | 9.0 ± | 4.9 | 9.1 ± 3.0 | 9.1 + | 2.4 | 9.0 + 4.2 | 9.0 + | 2.4 ^{b} |

| | | Mean | | | |
|---|---|---|---|---|---|
| Variable | Group | (SEM) | Effect | p-Level | Eta² |
| Correct Response | PLA | 97.3 ± 3.4 | Time | 0.505 | 0.041 |
| | PrX | 102.5 ± 3.4 | W × T | 0.578 | 0.033 |
| | Time | | G × T | 0.072 | 0.097 |
| Errors | PLA | 23.6 ± 3.9 | Time | 0.425 | 0.044 |
| | PrX | 18.0 ± 3.9 | W × T | 0.308 | 0.055 |
| | Time | | G × T | 0.255 | 0.061 |
| Perseverative Errors | PLA | 14.4 ± 1.2 | Time | 0.409 | 0.036 |
| (PEBL) | PrX | 12.7 ± 1.2 | W × T | 0.559 | 0.020 |
| | Time | | G × T | 0.369 | 0.041 |
| Perseverative Errors | PLA | 10.9 ± 1.6 | Time | 0.432 | 0.033 |
| (PAR rules) | PrX | 8.7 ± 1.6 | W × T | 0.571 | 0.019 |
| | Time | | G × T | 0.559 | 0.020 |

Data are expressed as means ± standard deviations for the placebo (PLA) and Paraxanthine (PrX).
Greenhouse-Geisser univariate p-levels are listed for time (T), weight × time (W × T) and group × time (G × T) interactions effects.
Pairwise comparison is indicated by the following superscripts:
^{†} = p < 0.05 difference from baseline value;
* = 0.05 < p < 0.10 difference from baseline;
^{a} = p < 0.05 difference between groups;
^{b} = 0.05 < p < 0.10 difference between groups.

### Go-No Go Test

A test of sustained attention and response control (i.e., attention/inhibition). Lower response times represents better ability to pay attention and inhibitory control.

Mean Correct Responses were 1.1% higher in the paraxanthine group compared to placebo (placebo 91.5% vs. paraxanthine 92.5%), with correct responses in the placebo group decreasing by 1.4% and paraxanthine by 0.5% in comparison to baseline. Mean response times were unchanged in the paraxanthine group (−0.3%), however declined by 3.5% in the placebo group. Lower response times represent better ability to pay attention and inhibitory control.

Paraxanthine administration resulted in preventing mental fatigue, by maintaining attention and inhibitory control in comparison to placebo for
Round 1 Response Time—Condition R Mean
Round 2 Response time for condition P mean
Average Response Time Mean R1 and T2 trials

Acute paraxanthine supplementation resulted in faster response times to correctly respond compared to PLA (shows less metal fatigue) in the go/no-go test. In addition, paraxanthine maintained percentage of correct answers, while placebo showed a significant decrease in correct answers. Paraxanthine increased the capacity for sustained attention and response control.

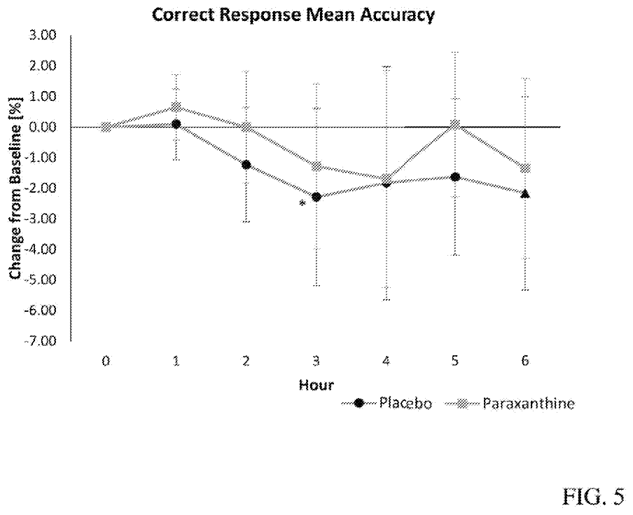

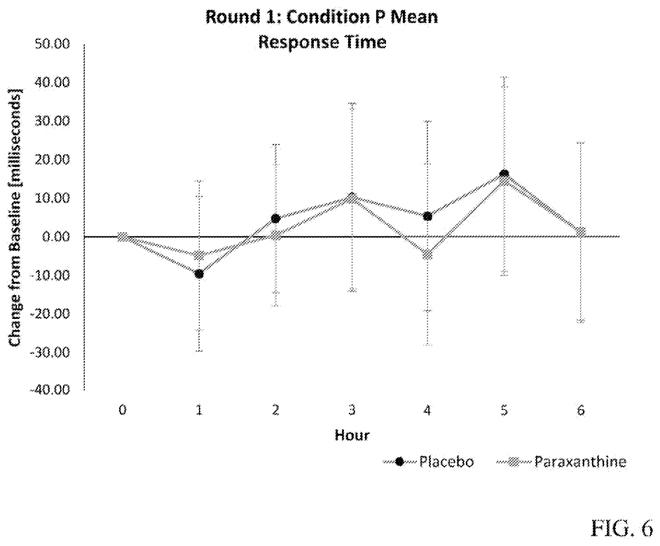

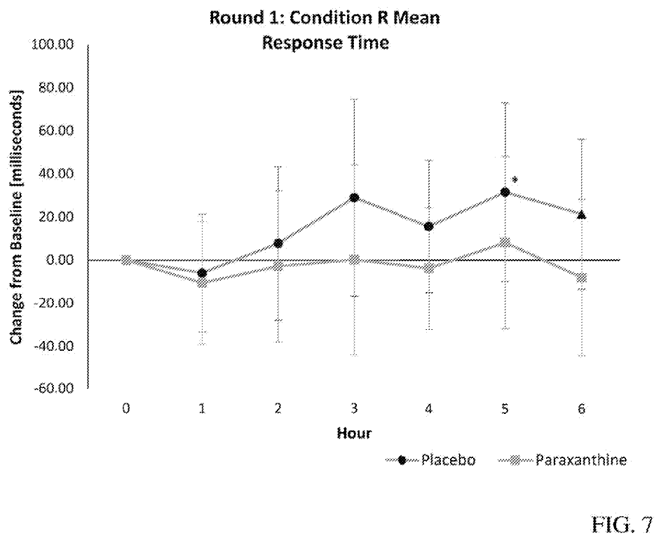

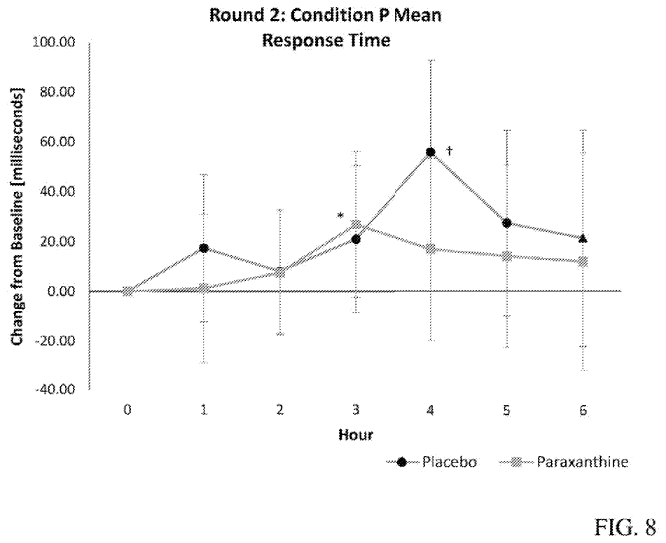

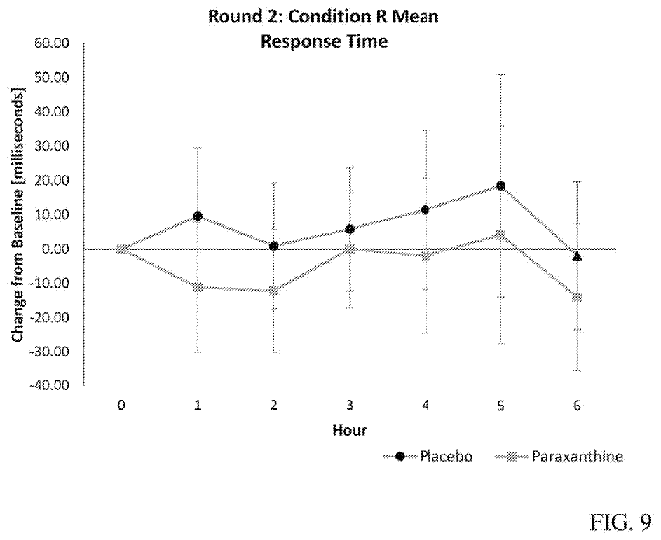

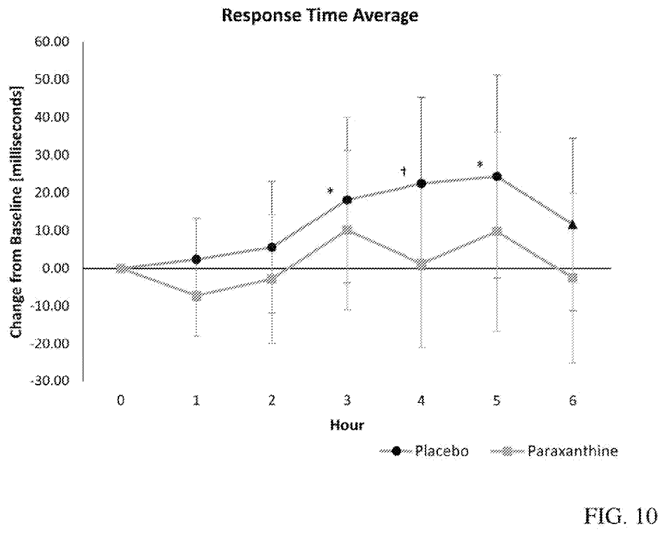

FIGS. 5-10 show various graphs of the Go/No-Go test results: FIG. 5 shows placebo significantly decreased number of correct responses (hour 3, p<0.5); FIG. 6 shows no significant differences over time or between treatments; FIG. 7 shows paraxanthine better maintained mean response time over time compared to PLA during round 1 of testing; FIG. 8 shows paraxanthine better maintained mean response time over time compared to PLA during round 2 of testing; FIG. 9 shows paraxanthine non-significantly better maintained mean response time over time compared to PLA during round 2 of testing; and FIG. 10 shows average response times significantly decreased in the placebo group, while paraxanthine maintained mean response time over time (round 1+round 2) (i.e., shows less fatigue).

TABLE 2
Results of the Go/No-Go Test
Go/No-Go

| | | Hour | | | | | | | | | | Mean | | | |
|---|---|---|---|---|---|---|---|---|---|---|---|---|---|---|---|
| Variable | Group | 0 | 1 | 2 | 3 | | 4 | | 5 | | 6 | (SEM) | Effect | p-Level | Eta² |
| Correct Response Mean | PLA | 92.8 + 4.9 | 92.9 ± 4.1 | 91.6 ± 5.8 | 90.6 ± | 8.0 | 91.0 ± | 7.2 | 91.2 ± | 5.6 | 90.7 ± 7.0 | 91.5 ± 1.6 | Time | 0.448 | 0.039 |
| Accuracy (%) | PX | 93.0 ± 2.9 | 93.7 ± 2.3 | 92.9 ± 3.6 | 91.7 ± | 4.6 | 91.3 ± | 6.9 | 93.1 ± | 3.9 | 91.6 ± 4.4 | 92.5 ± 1.3 | W × T | 0.206 | 0.067 |
| | Time | 92.9 ± 4.0 | 93.3 ± 3.3 | 92.2 ± 4.8 | 91.1 ± | 6.5 * | 91.2 ± | 6.9 | 92.1 ± | 4.9 | 91.1 ± 5.8 | | G × T | 0.851 | 0.012 |
| Round 1: Condition P Mean - | PLA | 377.99 + 72.77 | 368.45 + 104.38 | 382.78 + 69.69 | 388.27 + | 74.50 | 383.31 + | 85.85 | 394.18 + | 73.69 | 379.01 + 72.45 | 382.09 + 17.36 | Time | 0.531 | 0.035 |
| Response Time (Millisecond) | PrX | 392.29 ± 44.29 | 387.51 ± 38.34 | 392.71 ± 38.09 | 402.18 ± | 59.28 | 387.65 ± | 52.22 | 406.70 ± | 50.76 | 393.51 ± 53.02 | 394.55 ± 18.07 | W × T | 0.573 | 0.032 |
| | Time | 384.85 ± 60.00 | 377.60 ± 78.84 | 387.54 ± 55.85 | 394.94 ± | 66.60 | 385.40 ± | 70.28 | 400.19 ± | 62.74 * | 385.97 ± 62.98 | | G × T | 0.964 | 0.007 |
| Round 1: Condition R Mean - | PLA | 336.57 + 79.49 | 330.61 + 97.87 | 344.31 + 76.34 | 365.57 + | 58.10 | 352.13 + | 85.87 | 368.05 + | 78.25 * | 357.90 + 66.43 | 351.29 + 14.48 | Time | 0.211 | 0.065 |
| Response Time (Millisecond) | Time | 353.43 ± 59.53 | 342.90 ± 30.97 | 350.55 ± 32.17 | 353.66 ± | 55.84 | 349.53 ± | 43.93 | 361.56 ± | 44.42 | 345.33 ± 44.12 | 350.34 ± 15.74 | W × T | 0.220 | 0.064 |
| | PrX | 344.30 ± 70.08 | 336.24 ± 73.85 | 347.17 ± 59.16 | 360.11 ± | 56.16 | 350.94 ± | 68.47 | 365.08 ± | 63.74 | 352.14 ± 56.48 | | G × T | 0.775 | 0.023 |
| Round 2: Condition P Mean - | PLA | 319.45 ± 88.65 | 336.87 ± 43.09 | 327.47 ± 97.12 | 340.48 ± | 87.15 | 375.27 ± | 101.29 ^{†} | 346.77 ± | 57.01 | 340.70 ± 88.02 | 340.83 ± 18.00 | Time | 0.407 | 0.045 |
| Response Time (Millisecond) | PrX | 345.19 ± 37.11 | 346.39 ± 47.07 | 352.72 ± 46.18 | 372.09 ± | 74.77 * | 362.02 ± | 66.64 | 359.18 ± | 62.70 | 357.05 ± 99.19 | 356.55 ± 18.00 | W × T | 0.277 | 0.059 |
| | Time | 332.32 ± 67.75 | 341.63 ± 44.40 | 340.09 ± 75.48 | 356.29 ± | 81.04 ^{†} | 368.65 ± | 84.12 ^{†} | 352.98 ± | 58.95 | 348.87 ± 92.09 | | G × T | 0.588 | 0.031 |
| Round 2: Condition R Mean - | PLA | 377.57 ± 99.36 | 387.18 ± 65.21 | 378.51 ± 106.06 | 383.44 ± | 82.29 | 389.05 ± | 89.88 | 395.98 ± | 77.08 | 375.61 ± 97.28 | 384.01 ± 19.48 | Time | 0.190 | 0.067 |
| Response Time (Millisecond) | PrX | 401.46 ± 52.36 | 390.30 ± 41.57 | 389.31 ± 39.82 | 401.47 ± | 54.29 | 399.45 ± | 62.91 | 405.58 ± | 62.24 | 387.41 ± 53.47 | 396.32 ± 20.27 | W × T | 0.202 | 0.066 |
| | Time | 389.04 ± 79.63 | 388.68 ± 54.04 | 383.70 ± 79.88 | 392.10 ± | 69.43 | 394.04 ± | 76.69 | 400.59 ± | 69.07 | 381.27 ± 77.96 | | G × T | 0.845 | 0.015 |
| Response Time Average | PLA | 353.48 + 81.86 | 355.89 + 73.08 | 359.07 + 82.85 | 371.64 + | 70.69 * | 375.93 + | 85.41 ^{†} | 377.81 + | 65.15 * | 365.08 + 75.28 | 365.69 + 16.76 | Time | 0.684 | 0.025 |
| | PrX | 373.88 ± 43.64 | 366.68 ± 36.59 | 371.09 ± 30.25 | 384.07 ± | 51.07 | 375.03 ± | 52.38 | 383.60 ± | 47.82 | 371.27 ± 53.76 | 374.94 ± 17.45 | W × T | 0.625 | 0.028 |
| | Time | 363.27 ± 65.81 | 361.07 ± 57.57 | 364.84 ± 62.36 | 377.61 ± | 61.11* | 375.49 ± | 70.04 | 380.59 ± | 56.38 * | 368.05 ± 64.56 | | G × T | 0.760 | 0.020 |

Data are expressed as means ± standard deviations for the placebo (PLA) and Paraxanthine (PrX).
Greenhouse-Geisser univariate p-levels are listed for time (T), weight × time (W × T) and group × time (G × T) interactions effects.
Pairwise comparison is indicated by the following superscripts:
^{†} = p < 0.05 difference from baseline value;
* = 0.05 < p < 0.10 difference from baseline;
^{a} = p < 0.05 difference between groups;
^{b} = 0.05 < p < 0.10 difference between groups.

### Vigilance

The psychomotor vigilance task test is a sustained attention, reaction timed task that measures the speed with which participants respond to a visual stimulus. Lower Reaction time, especially maintained throughout this many tests represents better ability to sustain attention.

Paraxanthine had no effect on average reaction times in the vigilance task test.

Acute supplementation with paraxanthine resulted in sustained attention (maintained reaction times, prevention of mental fatigue) in the Vigilance Task Test, measuring a person's ability to remain heedfully vigilant. In contrast, placebo showed significantly reduced reactions times (overall hours 3 and 6).

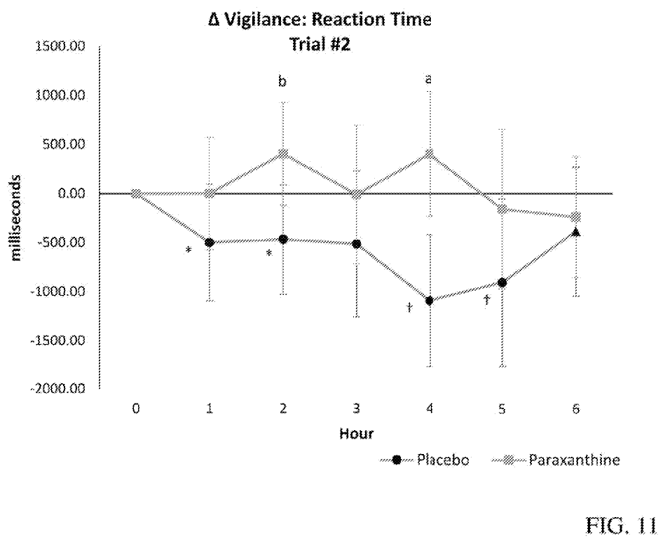

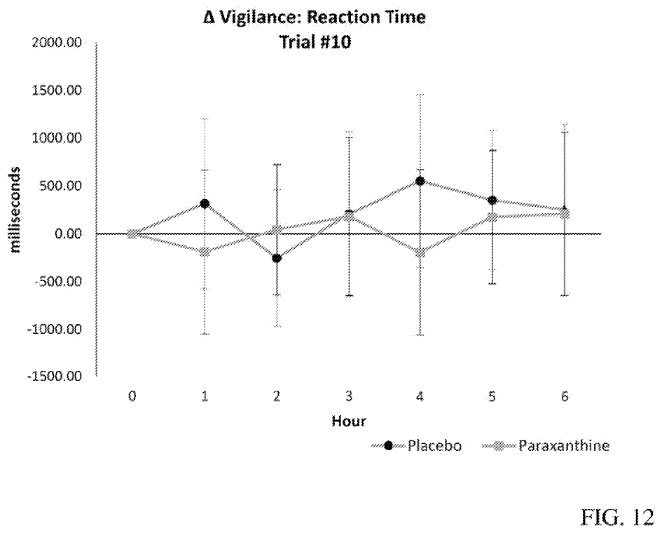

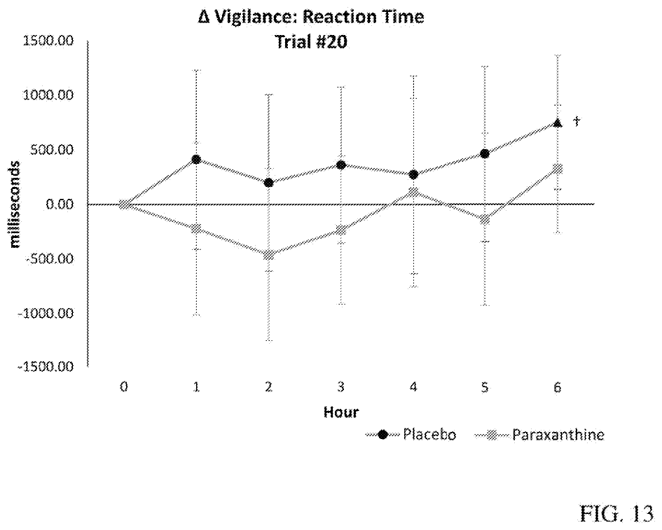

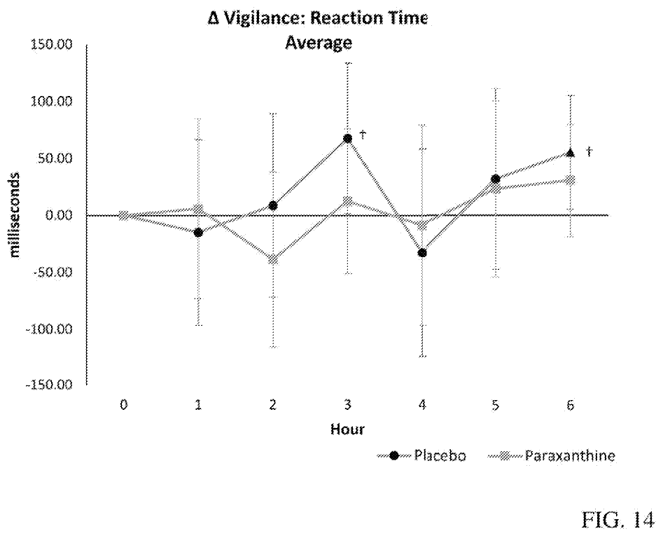

FIGS. 11-14 show various graphs of the Vigilance test results: FIG. 11 shows paraxanthine maintained reaction time compared to significant faster reaction times in the PLA treatment; FIG. 12 shows no significant time or between treatment differences; FIG. 13 shows reaction time increased over time in the PLA treatment but was maintained in the paraxanthine treatment (i.e., less fatigue); and FIG. 14 shows reaction time increased over time in the PLA treatment (showed a trend vs. baseline at hours 3 and 6, p<0.1) but was maintained in the paraxanthine treatment (i.e., less fatigue).

TABLE 3
Results of the Vigilance Test
Vigilance

| | | Hour | | | | | | | | | | | | | |
|---|---|---|---|---|---|---|---|---|---|---|---|---|---|---|---|
| Variable | Group | 0 | | 1 | | 2 | | 3 | | 4 | | 5 | | 6 | |
| Reaction Time | PLA | 3,005 ± | 1 | 2,506 ± | 952 * | 2,538 ± | 904 * | 2,492 ± | 919 | 1,912 ± | 1,233 ^{†} | 2,096 ± | 1,222 ^{†} | 2,614 ± | 954 |
| (milliseconds) | PrX | 2,601 ± | 950 | 2,602 ± | 943 | 3,005 ± | 1 | 2,591 ± | 967 | 3,005 ± | 1 | 2,441 ± | 1,025 | 2,360 ± | 1,168 |
| Trial #2 | Time | 2,811 ± | 675 | 2,552 ± | 929 | 2,762 ± | 682 ^{b} | 2,540 ± | 924 | 2,437 ± | 1,035 ^{a} | 2,261 ± | 1,122 * | 2,492 ± | 1,048 |
| Reaction Time | PLA | 2,295 ± | 1,121 | 2,612 ± | 958 | 2,042 ± | 1,116 | 2,501 ± | 965 | 2,848 ± | 569 | 2,646 ± | 689 | 2,546 ± | 878 |
| (milliseconds) | PrX | 2,607 ± | 929 | 2,418 ± | 1,076 | 2,651 ± | 839 | 2,788 ± | 751 | 2,412 ± | 1,081 | 2,783 ± | 769 | 2,813 ± | 665 |
| Trial #10 | Time | 2,445 ± | 1,025 | 2,519 ± | 1,000 | 2,335 ± | 1,020 | 2,639 ± | 864 | 2,639 ± | 864 | 2,712 ± | 716 | 2,674 ± | 779 |
| Reaction Time | PLA | 2,254 ± | 1,177 | 2,665 ± | 832 | 2,453 ± | 1,060 | 2,617 ± | 950 | 2,528 ± | 919 | 2,717 ± | 704 ^{†} | 3,005 ± | 1 |
| (milliseconds) | PrX | 2,680 ± | 767 | 2,458 ± | 1,005 | 2,220 ± | 1,172 | 2,445 ± | 1,021 | 2,792 ± | 739 | 2,544 ± | 898 | 3,005 ± | 1 |
| Trial #20 | Time | 2,458 ± | 1,005 | 2,566 ± | 906 | 2,341 ± | 1,098 | 2,535 ± | 968 | 2,655 ± | 831 | 2,634 ± | 790 ^{†} | 3,005 ± | 1 |
| Reaction Time | PLA | 2,535 ± | 124 | 2,521 ± | 114 | 2,544 ± | 147 | 2,603 ± | 158 ^{†} | 2,503 ± | 137 | 2,567 ± | 101 | 2,591 ± | 101 ^{†} |
| (milliseconds) | PrX | 2,546 ± | 55 | 2,552 ± | 122 | 2,508 ± | 201 | 2,559 ± | 140 | 2,538 ± | 186 | 2,570 ± | 84 | 2,577 ± | 96 |
| Average | Time | 2,541 ± | 95 | 2,536 ± | 117 | 2,527 ± | 172 | 2,582 ± | 148 * | 2,519 ± | 160 | 2,568 ± | 92 | 2,584 ± | 97 ^{†} |

| | | Mean | | | | |
|---|---|---|---|---|---|---|
| Variable | Group | (SEM) | | Effect | p-Level | Eta² |
| Reaction Time | PLA | 2,451 ± | 99 | Time | 0.837 | 0.017 |
| (milliseconds) | PrX | 2,659 ± | 103 | W × T | 0.824 | 0.018 |
| Trial #2 | Time | | | G × T | 0.124 | 0.076 |
| Reaction Time | PLA | 2,499 ± | 86 | Time | 0.982 | 0.007 |
| (milliseconds) | PrX | 2,639 ± | 90 | W × T | 0.997 | 0.003 |
| Trial #10 | Time | | | G × T | 0.508 | 0.038 |
| Reaction Time | PLA | 2,608 ± | 92 | Time | 0.240 | 0.059 |
| (milliseconds) | PrX | 2,590 ± | 96 | W × T | 0.311 | 0.052 |
| Trial #20 | Time | | | G × T | 0.708 | 0.025 |
| Reaction Time | PLA | 2,552 ± | 26 | Time | 0.923 | 0.009 |
| (milliseconds) | PrX | 2,550 ± | 27 | W × T | 0.827 | 0.015 |
| Average | Time | | | G × T | 0.659 | 0.026 |

Data are expressed as means ± standard deviations for the placebo (PLA) and Paraxanthine (PrX).
Greenhouse-Geisser univariate p-levels are listed for time (T), weight × time (W × T) and group × time (G × T) interactions effects.
Pairwise comparison is indicated by the following superscripts:
^{†} = p < 0.05 difference from baseline value;
* = 0.05 < p < 0.10 difference from baseline;
^{a} = p < 0.05 difference between groups;
^{b} = 0.05 < p < 0.10 difference between groups.

### Sternberg

A test of working memory, ability to make use of short-term memory. A lower reaction time means participants can access their working memory faster. The Sternberg Task test is a widely used paradigm for studying short-term/working memory (STM/WM) involving cognitive control processes.

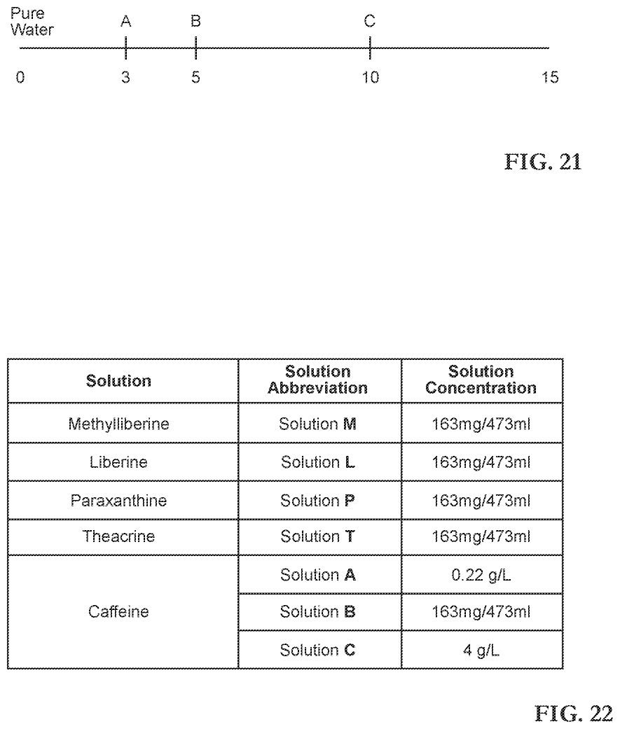

Overall, paraxanthine decreased mean response times in the Sternberg Test by 3.9% (faster) compared to baseline, whereas the placebo group was 2.7% faster. Paraxanthine significantly increased short-term/working memory in one measure of the Sternberg test (letter length 6, present reaction time after 4 and 5 hours, see FIG. 21). As the list length increases, probe judgments become less accurate and slower, indicating increases in short-term memory and working memory demands.

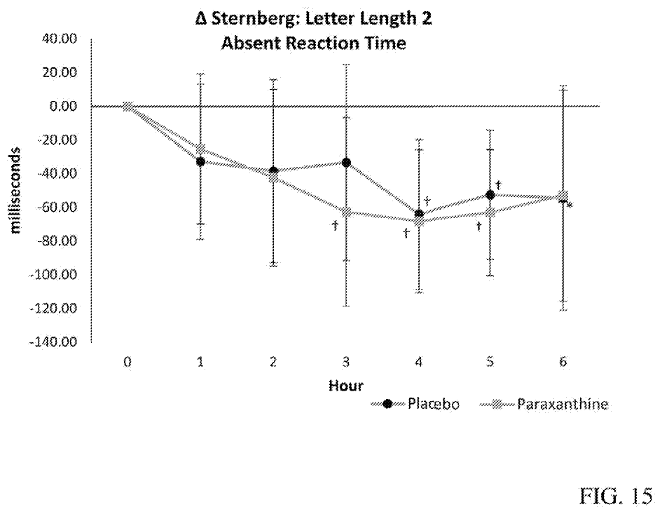

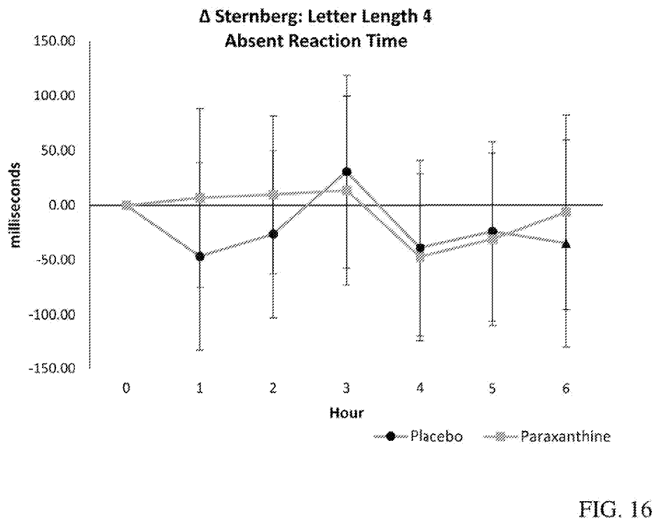

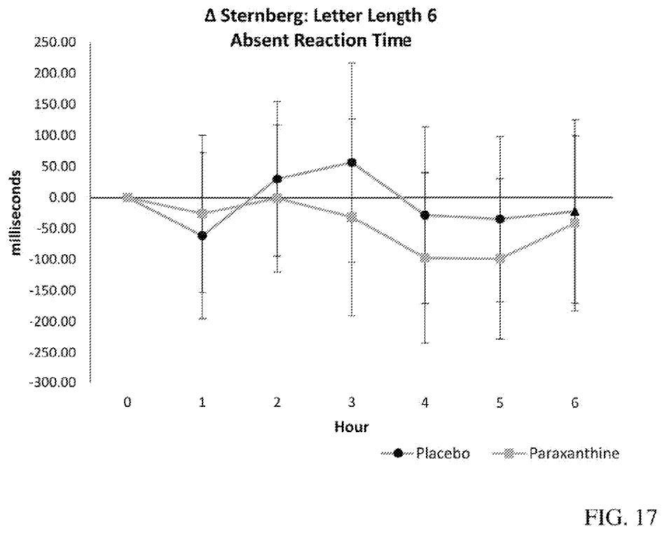

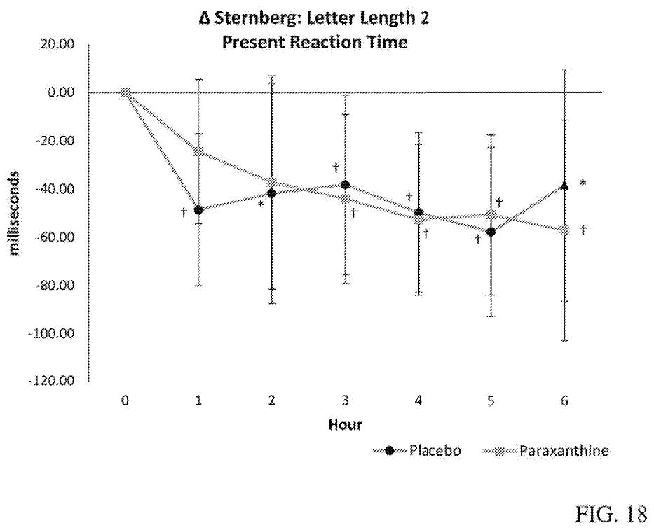

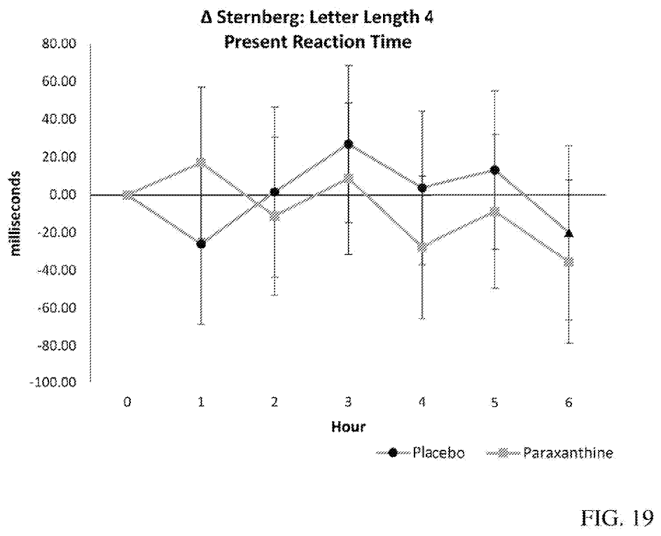

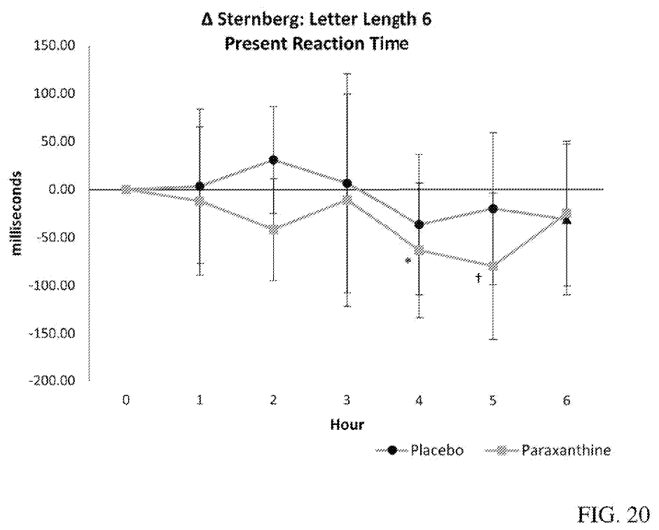

FIGS. 15-20 show various graphs of the results of the Sternberg test: FIG. 15 shows paraxanthine showed a trend towards improved reaction times at hour 3, 4 and 5, while placebo improved reaction time at hours 4 and 5 (p<0.1); FIG. 16 shows no significant differences over time or between treatments; FIG. 17 shows no significant differences over time or between treatments, with Paraxanthine showing greater improvement during hours 3-5; FIG. 18 shows paraxanthine and placebo significantly improved reaction times; FIG. 19 shows no significant differences over time or between treatments, with Paraxanthine showing greater improvements during hours 3-6; and FIG. 20 shows Paraxanthine improved present reaction time at later timepoints (hours 4 and 5), while the Placebo treatment did not.

TABLE 4
Results of Sternberg Test
Sternberg

| | | Hour | | | | | | | | | | | | | Mean | | | |
|---|---|---|---|---|---|---|---|---|---|---|---|---|---|---|---|---|---|---|
| Variable | Group | 0 | 1 | | 2 | | 3 | | 4 | | 5 | | 6 | | (SEM) | Effect | p-Level | Eta² |
| Letter Length 2: Absent | PLA | 620.6 ± 86.9 | 587.8 ± | 106.8 | 582.2 ± | 70.1 | 587.3 ± | 92.6 | 556.6 ± | 79.7 ^{†} | 568.1 ± | 91.1 ^{†} | 566.2 ± | 96.8 * | 580.7 ± 26.0 | Time | 0.382 | 0.046 |
| Reaction Time (Millisecond) | PrX | 621.9 ± 154.3 | 596.7 ± | 114.2 | 579.7 ± | 133.1 | 559.3 ± | 127.9 ^{†} | 553.8 ± | 107.0 ^{†} | 558.8 ± | 101.0 ^{†} | 568.9 ± | 128.3 | 577.6 ± 27.1 | W × T | 0.363 | 0.047 |
| | Time | 621.2 ± 121.2 | 592.0 ± | 108.1 * | 581.0 ± | 102.9 ^{†} | 573.8 ± | 109.5 ^{†} | 555.2 ± | 91.8 ^{†} | 563.6 ± | 94.1 ^{†} | 567.5 ± | 110.6 ^{†} | | G × T | 0.880 | 0.013 |
| Letter Length 4: Absent | PLA | 751.5 ± 134.9 | 704.9 ± | 98.3 | 725.2 ± | 95.1 | 782.3 ± | 188.6 | 712.5 ± | 90.1 | 727.5 ± | 127.4 | 716.7 ± | 125.1 | 731.1 ± 38.4 | Time | 0.337 | 0.050 |
| Reaction Time (Millisecond) | PrX | 748.7 ± 148.5 | 755.5 ± | 189.6 | 758.4 ± | 202.2 | 762.3 ± | 245.4 | 701.3 ± | 156.9 | 717.5 ± | 189.7 | 742.2 ± | 192.6 | 741.3 ± 40.0 | W × T | 0.185 | 0.067 |
| | Time | 750.2 ± 138.6 | 729.2 ± | 148.2 | 741.2 ± | 153.5 | 772.7 ± | 213.3 | 707.2 ± | 124.0 | 722.7 ± | 156.9 | 728.9 ± | 158.1 | | G × T | 0.684 | 0.026 |
| Letter Length 6: Absent | PLA | 947.5 ± 192.6 | 886.3 ± | 126.1 | 977.8 ± | 189.6 | 1,004.1 ± | 256.5 | 918.8 ± | 168.0 | 912.4 ± | 134.5 | 924.9 ± | 185.0 | 938.8 ± 53.5 | Time | 0.211 | 0.063 |
| Reaction Time (Millisecond) | PrX | 1,012.5 ± 334.2 | 986.5 ± | 293.1 | 1,011.3 ± | 287.6 | 980.9 ± | 362.1 | 914.8 ± | 206.2 | 913.5 ± | 191.6 | 970.7 ± | 236.6 | 970.0 ± 55.7 | W × T | 0.166 | 0.069 |
| | Time | 978.7 ± 266.1 | 934.4 ± | 223.5 | 993.8 ± | 237.0 | 993.0 ± | 305.2 | 916.8 ± | 183.3 | 912.9 ± | 160.8 | 946.9 ± | 208.1 | | G × T | 0.692 | 0.025 |
| Letter Length 2: Present | PLA | 557.2 ± 58.2 | 508.6 ± | 57.0 ^{†} | 515.5 ± | 52.7 * | 519.0 ± | 53.5 ^{†} | 507.5 ± | 57.8 ^{†} | 499.3 ± | 62.8 ^{†} | 518.7 ± | 77.0 * | 517.5 ± 17.1 | Time | 0.662 | 0.027 |
| Reaction Time (Millisecond) | PrX | 541.1 ± 104.2 | 516.6 ± | 79.6 | 503.8 ± | 97.0 | 497.1 ± | 98.2 ^{†} | 488.3 ± | 78.7 ^{†} | 490.3 ± | 66.2 ^{†} | 483.8 ± | 65.7 ^{†} | 503.5 ± 17.8 | W × T | 0.551 | 0.034 |
| | Time | 549.5 ± 82.1 | 512.4 + | 67.4 ^{†} | 509.9 ± | 75.8 ^{†} | 508.5 ± | 77.3 ^{†} | 498.3 ± | 67.9 ^{†} | 495.0 ± | 63.3 ^{†} | 502.0 ± | 72.6 ^{†} | | G × T | 0.657 | 0.027 |
| Letter Length 4: Present | PLA | 634.2 ± 72.9 | 608.2 ± | 63.9 | 635.8 ± | 80.2 | 661.2 ± | 93.5 | 637.9 ± | 84.6 | 647.2 ± | 135.5 | 614.1 ± | 95.1 | 633.8 ± 24.2 | Time | 0.486 | 0.039 |
| Reaction Time (Millisecond) | PrX | 625.9 ± 100.6 | 642.9 ± | 135.2 | 614.7 ± | 151.3 | 634.6 ± | 123.2 | 598.2 ± | 100.0 | 617.1 ± | 96.6 | 590.3 ± | 86.5 | 617.9 ± 25.1 | W × T | 0.277 | 0.055 |
| | Time | 630.2 ± 85.5 | 624.9 ± | 103.6 | 625.6 ± | 117.6 | 648.4 ± | 107.3 | 618.8 ± | 92.6 | 632.8 ± | 117.0 | 602.7 ± | 90.0 * | | G × T | 0.445 | 0.041 |
| Letter Length 6: Present | PLA | 781.5 ± 82.3 | 784.8 ± | 89.0 | 812.3 ± | 136.7 | 788.2 ± | 170.2 | 744.8 ± | 67.9 | 761.4 ± | 99.4 | 750.2 ± | 88.1 | 774.9 ± 38.6 | Time | 0.880 | 0.012 |
| Reaction Time (Millisecond) | PrX | 841.6 ± 254.0 | 829.7 ± | 178.1 | 800.0 ± | 196.0 | 830.6 ± | 260.2 | 778.1 ± | 177.4 * | 761.8 ± | 149.9 ^{†} | 816.4 ± | 187.9 | 808.2 ± 40.2 | W × T | 0.896 | 0.011 |
| | Time | 810.4 ± 184.1 | 806.4 ± | 137.9 | 806.4 ± | 164.3 | 808.5 ± | 214.4 | 760.8 ± | 130.4 * | 761.6 ± | 123.5 * | 782.0 ± | 145.6 | | G × T | 0.665 | 0.026 |
| Mean Response Time | PLA | 715.4 ± 85.3 | 680.1 ± | 71.2 | 708.1 ± | 74.8 | 723.7 ± | 118.1 | 679.7 ± | 64.9 | 686.0 ± | 90.8 | 681.8 ± | 84.5 | 696.1 ± 31.1 | Time | 0.310 | 0.052 |
| | PrX | 731.9 ± 163.0 | 721.3 ± | 152.2 | 711.3 ± | 163.0 | 710.8 ± | 188.4 | 672.4 ± | 124.5 ^{†} | 676.5 ± | 121.0 ^{†} | 695.4 ± | 136.5 | 703.1 ± 32.4 | W × T | 0.225 | 0.062 |
| | Time | 723.4 ± 126.0 | 699.9 ± | 116.6 | 709.7 ± | 122.4 | 717.5 ± | 152.6 | 676.2 ± | 96.0 ^{†} | 681.4 ± | 104.2 ^{†} | 688.3 ± | 110.3 * | | G × T | 0.533 | 0.034 |

Data are expressed as means ± standard deviations for the placebo (PLA) and Paraxanthine (PrX).
Greenhouse-Geisser univariate p-levels are listed for time (T), weight × time (W × T) and group × time (G × T) interactions effects.
Pairwise comparison is indicated by the following superscripts:
^{†} = p < 0.05 difference from baseline value;
* = 0.05 < p < 0.10 difference from baseline;
^{a} = p < 0.05 difference between groups;
^{b} = 0.05 < p < 0.10 difference between groups.

### Toxicity

In order to assess the relative toxicity between paraxanthine and caffeine and other caffeine metabolites, toxicology studies were performed and LD₅₀ was determined. LD₅₀ for paraxanthine to be 1,601 mg/kg of body weight. In contrast, the LD50 of caffeine is 192 mg/kg, suggesting that caffeine has significantly greater levels of toxicity in than paraxanthine alone. Furthermore, the LD50 for of the caffeine metabolites theobromine and theophylline are 1,265 mg/kg and 225 mg/kg, respectively. Taken together, these results indicate paraxanthine has a substantially superior safety profile than caffeine or its other major metabolites.

### Taste

In order to assess palatability of paraxanthine relative to methylliberine, liberine, and theacrine, a series of blind taste tests was performed. Subjects were trained prior to the blind taste tests to distinguish tastes and to use the scale set forth in FIG. 21. Subjects were given Solutions A and C, as set forth in FIG. 22 and informed of their bitterness scores (Solution A=3, Solution C=10). Subjects were then given Solution B and discussed the consistent scores.

During training sessions the subjects were administered Solutions A and C for memory and then scored Solution B to memorize the scale. Subject were then given solutions of various caffeine concentrations to further memorize the scale.

After four training sessions were completed the subjects were administered the blind taste test, where subjects scored the bitterness of four product solutions—Solution L (Liberine), Solution P (Paraxanthine), Solution T (Theacrine), and Solution M (Methylliberine) on the scale established by the caffeine solutions. Each of the Solutions was administered at a concentration of 163 mg/473 ml, as shown in FIG. 22.

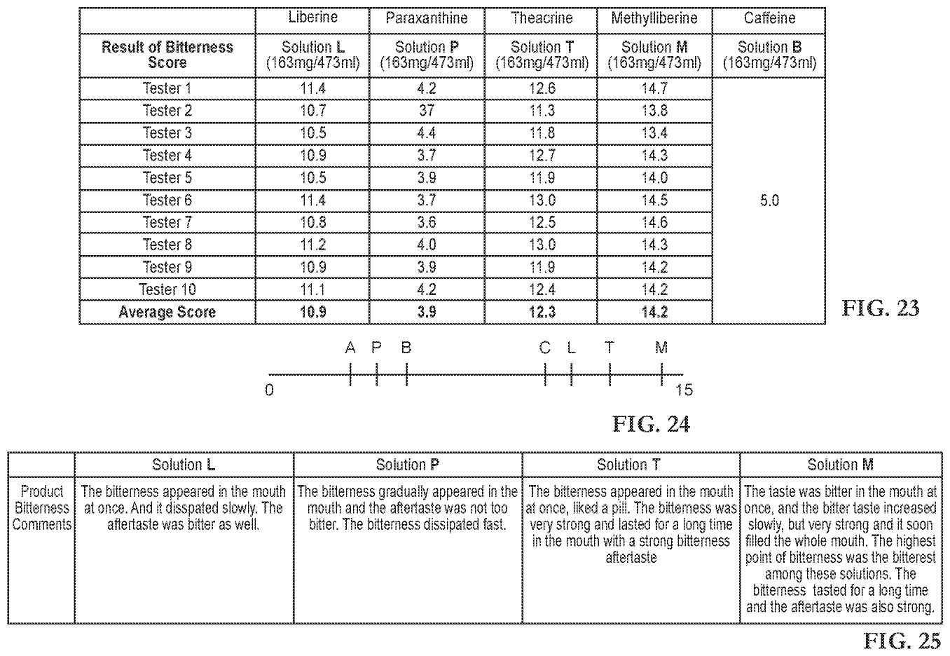

Subjects were administered Solutions L, P, T and M blindly and in a randomized order and scored each solution on the scale established by the caffeine solutions. FIGS. 23 and 24 show the results of the scoring, showing that tasters rated paraxanthine as substantially less bitter than solutions of equal concentration of methylliberine, liberine, and theacrine. Further, comments from the tasters are noted in FIG. 25.

### Paraxanthine Pre-Workout

Health subjects were administered paraxanthine and asked to report their experience. Subjects reported the following.

Male, 46, works out 4-5× week, uses coffee or energy drinks prior to workouts and might use coffee, energy drink or diet soda in the afternoon. Subject reported: “I like it . . . much better than my caffeine crutch. It feels different. The best way to describe it I feel happier, better, more energy and focused, but without being “stimulated” or “dizzy” or “anxious” just clean energy. I really did feel it.”

Male 38, likes nootropic products like piracetam, alpha-GPC, noopept, and has lots of experience with brain boosters. Subject reported: “I am surprised. I tried it alone and it was phenomenal . . . I loved it, but it was even better . . . like in a synergistic way, with my nootropic stack. I am sold. Please tell me when this is available.”

Female 44, nurse who plays sand volleyball twice weekly. Subject reported: “I loved this. It felt a bit different than my usual caffeine sources. I had clean energy, but not stressed or stimmed. I also slept great. Sometimes caffeine really disturbs my sleep quality and has me waking up several times during the night.”

Female, 19, college student, active, volleyball player. Subject reported: “I felt it. I love it. I want it. Simple. Bottom line it works. I don't like doing high caffeine as it just makes me feel crappy. This was great.”

Although the disclosure has been described with reference to preferred embodiments, persons skilled in the art will recognize that changes may be made in form and detail without departing from the spirit and scope of the disclosed apparatus, systems and methods.

Although the disclosure has been described with reference to preferred embodiments, persons skilled in the art will recognize that changes may be made in form and detail without departing from the spirit and scope of the disclosed apparatus, systems and methods.

What is claimed is:

1. A dietary supplement comprising a first active ingredient comprising about 2 mg to about 800 mg paraxanthine, either natural or synthetic.

2. The dietary supplement of claim 1, further comprising a second active ingredient, selected from a group consisting of: gallic acid, (+)-catechin (C), (−)-epicatechin (EC), (+)-gallocatechin (GC), (−)-epigallocatechin (EGC), (−)-catechin gallate (CG), (−)-gallocatechin gallate (GCG), (−)-epicatechin gallate (ECG) and (−)-epigallocatechin gallate (EGCG), glycerides, propylene glycol, lauroyl macrogol, lauroyl macrogol derivatives, co-crystallization products of bioperine, piperine, black pepper, bergamottin, dihydroxybergamottin (CYP3A4), flavonoids (naringin, hesperidin, nobiletin, tangeretin, quercetin), pterostilbene, fisetin, phytosomes, salicin, fish oil (omega-3 fatty acids and specialized, small lipid pro-resolving epoxide derivatives), oxylipins, tart cherry, hill oil, astaxanthin, proteolytic enzymes, glucosamine sulfate, chondroitin sulfate, MSM (methylsulfonylmethane), SAMe (S-adenosylmethionine), ASU (avocado-soybean unsapponifiable fraction), cetyl myristoleate, Dolichos falcate, triterpenoids, acacia catechu, Andrographis paniculata, Scutalleria baicalensis, Agmatine sulfate, Stinging Nettle, Sea Buckthorn, Curcumin, Cissus Quadrilangularis, Boswellia Serrata, Wasabia japonica (wasabi extract for Tea Tree Oil), Emu Oil, Arnica, Mangifera indica L. (Anacardiaceae), Lagenaria breviflora, Zingiber officinale (ginger & gingerols/shogaols), hoodia gordonii, caffeine, yohimbine, methylsynephrine, synephrine, theobromine, flavenoids, tocopherols, theophylline, alpha-yohimbine, conjugated linoleic acid (CLA), octopamine, evodiamine, passion flower, red pepper, cayenne, raspberry ketone, guggul, green tea, guarana, kola nut, beta-Phenethylamines, Acacia rigidula, forskolin (Coleus forskohlli), theophylline, synephrine, yohimbine, rhodiola, ashwagandha, ginseng, ginkgo Biloba, siberian ginseng, astragalus, licorice, green tea, reishi, dehydroepiandrosterone (DHEA), pregnenolone, tyrosine, N-acetyl-tyrosine, glucuronolactone, taurine, Acetyl-L-carnitine, 5-hydroxytryptophan, tryptophan, Phenethylamines, Sceletium tortuosum (and Mesembrine alkaloids), Dendrobium sp., Acacia rigidula, PQQ (Pyroloquinoline quinone), Ubiquinone(01), Nicotinamide riboside, picamilon, Huperzine A (Chinese clubmoss or Huperzia serrata, L-dopa, Mucuna pruriens, and forskolin (Coleus forskohlli), 2-(dimethylamino)ethanol (DMAE), DMAE bitartrate, and combinations thereof.

3. The dietary supplement of claim 1, further comprising a pharmaceutically acceptable carrier.

4. The dietary supplement of claim 1, wherein the supplement is in a solid oral dosage form.

5. The dietary supplement of claim 1, wherein the supplement is in a topical form for administration.

6. The dietary supplement of claim 1, further comprising a paraxanthine congener or paraxanthine analog.

7. The dietary supplement of claim 6, wherein said paraxanthine congener or analog is selected from the group consisting of caffeine, methyl caffeine, theobromine, theophylline, liberine, methylliberine, and combinations thereof.

8. The dietary supplement of claim 7, wherein the paraxanthine congener or analog is caffeine.

9. The dietary supplement of claim 8, wherein the effective dose of caffeine is lower than the effective dose of caffeine in a composition without paraxanthine.

10. A method for improving physical performance or energy in subject, comprising: providing the subject with a composition comprising about 2 mg to about 800 mg of paraxanthine, either natural or synthetic.

11. The method of claim 10, wherein upon administration of the composition, the subject experiences improvement of at least one of mood, energy, focus, concentration or sexual desire or a reduction of at least one of anxiety, fatigue, perception of effort or perception of pain.

12. The method of claim 11, wherein upon continued administration to the subject, the composition does not create dependence in the subject and/or withdrawal effect in the subject when continued use is ceased.

13. The method of claim 10, wherein the amount of paraxanthine provided is about 50 mg to about 400 mg.

14. The method of claim 10, wherein the subject experiences a decrease in fatigue of at least about 6 percent.

15. The method of claim 10, wherein the subject experiences an increase in energy of at least about 8 percent.

16. The method of claim 10, wherein the composition further comprises at least one ingredient selected from the group consisting of caffeine, theobromine, naringin, hesperidin, 2-(dimethylamino)ethanol (DMAE), DMAE bitartrate, and huperzine A.

17. A method of treating a condition in a subject in need thereof, comprising administering to the subject a composition comprising paraxanthine and a pharmaceutically acceptable carrier thereof.

18. The method of claim 17, wherein the condition is selected from narcolepsy, epilepsy, attention deficit disorders, attention deficit hyperactivity syndrome (ADHD), cognitive deficit disorders, palsies, uncontrolled anger, migraine, substance abuse addictions, eating disorders, depression, anxiety disorders, traumatic head injury (TBI), Parkinson's disease, Alzheimer's, and dementia.

19. The method of claim 18, wherein the paraxanthine is present from about 2 mg to about 800 mg.

20. The method of claim 18, wherein the composition is administered in a therapeutically effective amount.
